(19) United States
(12) Patent Application Publication
(10) Pub. No.: US 2006/0204462 A1
(43) Pub. Date: Sep. 14, 2006

(54) Michael addition product and Schiff's base aromachemicals

(76) Inventor: Luca Turin, London (GB)

Correspondence Address: MILES & STOCKBRIDGE PC, 1751 PINNACLE DRIVE, SUITE 500, MCLEAN, VA, 22102-3833, US

(21) Appl. No.: 11/335,826

(22) Filed: Jan. 20, 2006

Related U.S. Application Data
(60) Provisional application No. 60/651,078, filed on Feb. 9, 2005.
(60) Provisional application No. 60/661,505, filed on Mar. 15, 2005.
(60) Provisional application No. 60/668,107, filed on Apr. 5, 2005.
(60) Provisional application No. 60/724,778, filed on Oct. 11, 2005.

(51) Int. Cl. A61K 8/49; C07D 309/30; C07C 249/02

(52) U.S. Cl. 424/65; 510/101; 549/293; 549/321; 564/277

(57) ABSTRACT

A pro-odorant or pro-flavorant having the formula (I): 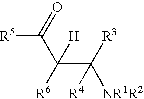
wherein R¹ is H, an aliphatic group or an aromatic group, R² is an aliphatic group or an aromatic group, provided that the total number of carbon atoms in the groups R¹ and R² is 10 or more; R³, R⁴ and R⁶ are each independently hydrogen or organic moieties which together with C(O)R⁵ render a compound of formula R⁵C(O)R⁶C=CR³R⁴ a material having odorant or flavourant characteristics and R⁵ is hydrogen or an organic moiety; or having the formula (II):
R⁸R⁹C=NR¹⁰
wherein R¹⁰ is an aliphatic group or an aromatic group having at least 10 carbon atoms; R⁸ and R⁹ are each independently hydrogen or organic moieties which together with C=O render a compound of formula R⁸R⁹C=O a material having odorant or flavourant characteristics, provided that only one of R⁸ and R⁹ is hydrogen; or having the formula (III): 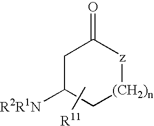
wherein R¹ is H, an aliphatic group or an aromatic group, R² is an aliphatic group or an aromatic group, provided that the total number of carbon atoms in the groups R¹ and R² is 10 or more; Z is CH₂ or O, n is 0 or 1, such that the ring is a 5 or 6 membered ring, R¹¹ is alkyl or alkenyl having up to 10 carbon atoms so as to render a compound of formula

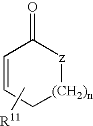

The present invention relates generally to the field of fragrances and flavorants. More particularly, the present invention relates to novel pro-fragrances and pro-flavorants that release their fragrance and flavor characteristics over extended times and their use as fragrance and flavor compositions for application to a variety of substrates.

Perfumed and flavored products, compositions and articles are well known in the art and widely used. The acceptance of these products by consumers depends to a large extent, however, on the ability thereof to retain and release their fragrance and/or flavor over time. The inability of certain perfume components to maintain their fragrance characteristics for acceptable lengths of time has been an ongoing problem in the area of fabric care. Perfume additives have traditionally been included in fabric detergents, bleaching compositions and fabric softeners. Typically, however, such perfumes are quickly lost to the fabric treatment environment, e.g. the wash and rinse water cycles in washing machines and the drying cycles in clothes dryers and little if any is imparted to or remains long on the treated fabric. Such inefficient delivery of expensive perfume components in fabric treatments results in high costs to manufacturers and consumers alike and has an adverse effect on consumer acceptance of such products.

The desire to extend the fragrance- and flavor-releasing properties over time of fragrance and flavor compositions exists in other industries and applications as well. For example, the consumer acceptance of numerous products would be greatly increased and their costs greatly reduced if the fragrance and flavor components incorporated therein could be made to last longer. Such products include foods, personal care products, household and industrial cleaning compositions, disinfectants, beverages, chewing gums, pharmaceutical and medicinal compositions and orally-deliverable matrices.

Numerous efforts have been made to improve the “lasting” properties of fragrances, particularly in the fabric care industry

U.S. Pat. No. 5,188,753 describes carrier mechanisms, such as encapsulation, which has not proven to be successful.

Other solutions involve the preparation of reaction products of the perfume with reactants to form products (pro-fragrances) that provide a delayed release of the fragrance over a longer period of time than by the use of the fragrance itself. See U.S. Patent Application Publication Nos. 20030211963, 20030211960, 20030134772, 20030073607, 20050043205, 20050009727, 20040147426, 20040116320, 20040097397, 20040018955 and U.S. Pat. Nos. 6,858,575; 6,790,815; 6,764,986; 6,740,713; 6,699,823; 6,566,312; 6,511,948; 6,451,751 and 6,413,920. Although providing compositions with pro-fragrance characteristics, the reaction of the fragrance compound with the reagents identified in the above-mentioned patents and publications provide reaction products that are either solids or comprise fluids with high viscosities.

The present invention relates particularly to the products of reactions between flavorant/fragrance aldehydes and ketones and a primary and/or secondary amine or an amino functional polymer comprising at least one primary and/or secondary amine group.

It is known to prepare reaction products between a variety of ketones and primary and/or secondary amines or amino functional polymers comprising at least one primary and/or secondary amine group. See, for example, U.S. Pat. Nos. 6,699,823; 6,511,948; 6,413,920 and 6,566,312, the entire contents and disclosures of which are incorporated herein by reference.

The listing or discussion of a prior-published document in this specification should not necessarily be taken as an acknowledgement that the document is part of the state of the art or is common general knowledge.

It has been suggested in the prior art to form Schiff bases of aldehydic fragrance compounds or aromachemicals. However, in such cases volatile amines such as alkyl anthranilates have been utilized that impart their own, sometimes objectionable, odor to the environment when the Schiff base degrades, thereby adversely impacting on the fragrance characteristics of the aromachemical.

It is an object of the present invention to provide novel products of reactions between aromachemical aldehydes and ketones and primary and/or secondary amines or amino functional polymers comprising at least one primary and/or secondary amine group.

Novel fragrance and flavor aromachemicals are disclosed herein as well as methods of making the compounds, uses of the aromachemicals and articles of manufacture including the aromachemicals. These novel derivatives find utility in any and all applications requiring certain aroma themes. The invention also relates to mixtures of these derivatives, methods for their preparation and their use as perfume materials for application to a variety of substrates. Examples of articles of manufacture in which the compounds of the invention may be incorporated include perfumes and colognes, candles, air fresheners, detergent compositions and disinfectants.

The present invention provides a pro-odorant or pro-flavorant having the formula (I): 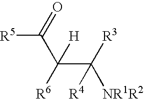
wherein R¹ is H, an aliphatic group or an aromatic group, R² is an aliphatic group or an aromatic group, provided that the total number of carbon atoms in the groups R¹ and R² is 10 or more; R³, R⁴ and R⁶ are each independently hydrogen or organic moieties which together with C(O)R⁵ render a compound of formula R⁵C(O)R⁶C=CR³R⁴ a material having odorant or flavourant characteristics and R⁵ is hydrogen or an organic moiety.

The groups R³ , R⁴, R⁵ and R⁶ may for example be H, linear or branched or cyclic C₁₋₁₂ alkyl, alkenyl or alkoxy, substituted linear or branched or cyclic C₁₋₁₂ alkyl, alkenyl or alkoxy, substituted or unsubstituted aromatic, halo, hydroxyl, thiol, thioether, amine, carboxylic acid, ester, nitro, cyano, isocyano, sulfonic acid, urea and thiourea. Suitable substituents on the substituted alkyl, alkenyl, alkoxy or aromatic groups include halo, hydroxyl, thiol, thioether, amine, carboxylic acid, ester, nitro, cyano, isocyano, sulfonic acid, urea and thiourea.

Preferably R⁵ and R³ are each independently linear or branched C₁₋₁₂ alkyl or alkenyl, substituted linear or branched C₁₋₁₂ alkyl or alkenyl, more preferably linear or branched C₁₋₆ alkyl or alkenyl. Still more preferably, R⁵ is an alkyl group containing four carbon atoms and most preferably R is —CH(CH₃)CH₂CH₃ or —CH₂CH₂CH=CH₂. R³ is preferably a methyl group.

Preferably at least one of R⁴ and R⁶ is H. For example, both of R⁴ and R⁶ are H. Another preferred group for R⁶ is cyclohexyl.

In one aspect of the invention R¹ is H, alkyl, alkenyl or alkoxy, R² is alkyl, alkenyl or alkoxy, provide that the total number of carbon atoms in the groups R¹ and R² is 10 or more, for example 12 to 25, or 15 to 20, for example 18. The alkyl, alkenyl or alkoxy group may be straight chained or branched or cyclic. More preferably R¹ is H. Alternatively, at least one of R¹ and R² may be an aromatic group.

Particularly preferred compounds of formula (I) include: 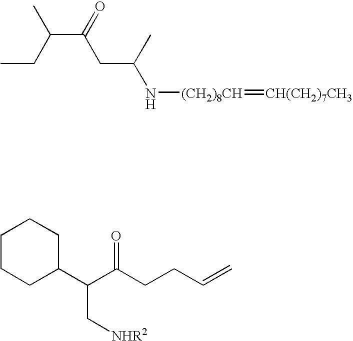
and

The present invention also provides a pro-odorant or pro-flavorant having the formula (II):
R⁸R⁹C=NR¹⁰
wherein R¹⁰ is an aliphatic group or an aromatic group having at least 10 carbon atoms; R⁸ and R⁹ are each independently hydrogen or organic moieties which together with C=O render a compound of formula R⁸R⁹C=O a material having odorant or flavourant characteristics, provided that only one of R⁸ and R⁹ is hydrogen.

The groups R⁸ and R⁹ may for example be H, linear or branched or cyclic C₁₋₁₂ alkyl, alkenyl or alkoxy, substituted linear or branched or cyclic C₁₋₁₂ alkyl, alkenyl or alkoxy, substituted or unsubstituted aromatic, halo, hydroxyl, thiol, thioether, amine, carboxylic acid, ester, nitro, cyano, isocyano, sulfonic acid, urea and thiourea. Suitable substituents on the substituted alkyl, alkenyl, alkoxy or aromatic groups include aromatic groups, halo, hydroxyl, thiol, thioether, amine, carboxylic acid, ester, nitro, cyano, isocyano, sulfonic acid, urea and thiourea.

Preferably R⁸ and R⁹ are each independently hydrogen, linear or branched or cyclic C₁₋₁₂ alkyl, alkenyl or alkoxy, substituted linear or branched or cyclic C₁₋₁₂ alkyl, alkenyl or alkoxy, substituted or unsubstituted aromatic, more preferably linear or branched C₁₋₆ alkyl or alkenyl. For example R⁸ may be an alkyl group containing four carbon atoms such as CH(CH₃)CH₂CH₃ or may contain an aromatic group, for example CH₂CH(CH₃)(C₆H₅). For example R⁹ may be hydrogen or a methyl group.

In one aspect of the invention R¹⁰ has at least 10 carbon atoms, for example 12 to 25, or 15 to 20, for example 18 and is alkyl, alkenyl or alkoxy. The alkyl, alkenyl or alkoxy group may be straight chained or branched.

A preferred compound of formula (II) is:

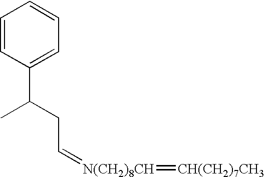

The present invention also provides a pro-odorant or pro-flavorant having the formula (III): 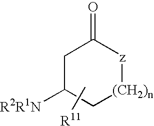
wherein R¹ is H, an aliphatic group or an aromatic group, R² is an aliphatic group or an aromatic group, provided that the total number of carbon atoms in the groups R¹ and R² is 10 or more; Z is CH₂ or O, n is 0 or 1, such that the ring is a 5 or 6 membered ring, R¹¹ is alkyl or alkenyl or alkoxy having up to 10 carbon atoms so as to render a compound of formula 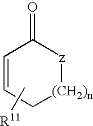
a material having odorant or flavorant characteristics.

In one aspect of the invention R¹ is H, alkyl, alkenyl or alkoxy, R² is alkyl, alkenyl or alkoxy, provide that the total number of carbon atoms in the groups R¹ and R² is 10 or more, for example 12 to 25, or 15 to 20, for example 18. The alkyl, alkenyl or alkoxy group may be straight chained or branched. More preferably R¹ is H. Alternatively, at least one of R¹ and R² may be an aromatic group.

R¹¹ may be a straight chain or branched alkyl or alkenyl or alkoxy. Optionally, R¹¹ may be substituted. Suitable substituents include halo, hydroxyl, thiol, thioether, amine, carboxylic acid, ester, nitro, cyano, isocyano, sulfonic acid, urea and thiourea.

Compounds of formula (III) in which Z is O and/or n is 0 are preferred.

Particularly preferred compounds of formula (III) include:

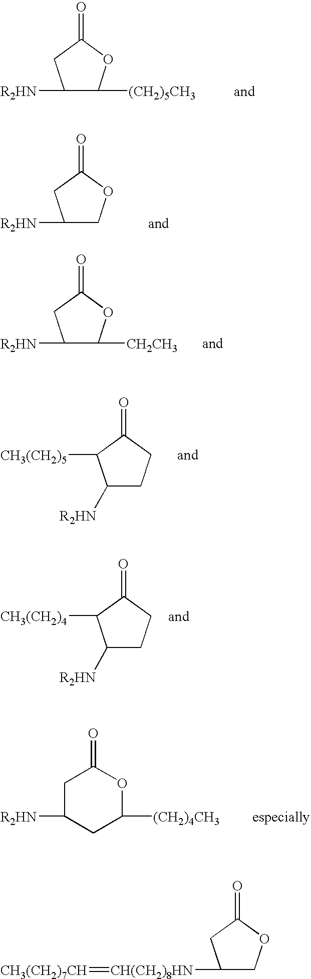

As used herein, by the terms “pro-odorant” and “pro-flavorant” we mean compounds which themselves have substantially no odor or flavor but degrade over time and/or under certain conditions to provide the odorant or flavorant molecule on which they are based and thus provide the odor or flavor of that base molecule.

The compounds of the invention of formula (I) may be produced by a Michael addition reaction of an aromachemcial comprising a carbon-carbon double bond in 1,4-conjugation with an aldehyde or ketone group such as an α, β-unsaturated ketone with a primary or secondary amine. The aromachemcials suitable for use in this reaction have the formula (IV): 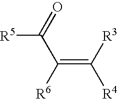
wherein R³, R⁴, R⁵ and R⁶ are each independently hydrogen or an organic moiety as defined above.

The amines used in this reaction have the formula NHR¹R² wherein R¹ and R² are as defined above.

Aromachemical/flavorants of formula (IV) that may be used in this reaction include but are not limited to:

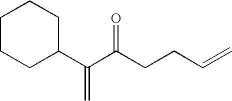

2-cyclohexyl-1,6-heptadien-3-one (Pharaone) (green, pineapple, fruity) and

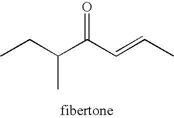

Filbertone is a known flavorant/fragrance compound possessing a characteristic hazelnut flavor/odor. Filbertone [chemical name—(E)-5-methyl-2-hepten-4-one; CAS Registry Number 102322-83-8 REGISTRY].

Many compounds wherein a double bond is 1,4-conjugated to a keto group have aroma and flavor characteristics.

Filbertone may be prepared according to the methods described in J. Braz. Chem. Soc., Vol. 9, No. 6, 583-585, 1998 and J. Jauch, et al, Angew. Chem., 101 (1989) 1039; Angew. Chem. Int. Ed. Engl., 28 (1989) 1022; J. Am. Chem. Soc. 61, 3303 (1939); Tetrahedron Letters 23, 335 (1982). The E-ketone has two stereoisomers:

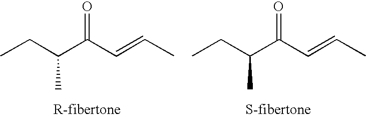

The R-stereoisomer possesses a hazelnut, soft, butter, chocolate, metallic flavor having an odor threshold about 10 times higher than for the S-stereoisomer. The R-stereoisomer is also known as (−)-(E,R)- Hazeltone or (−)-(E,R)-5-methyl-2-hepten-4-one.

The S-stereoisomer possesses a hazelnut, metallic, fatty, pyidine flavor having an odor threshold about 10 times lower than for the R-stereoisomer. It is also known as (+)-(E,S)-Hazeltone or (+)-(E,S)-5-methyl-2-hepten-4-one. The enantiomeric composition of raw hazelnuts falls in the ratio of 80-85% (+)-(E,S) and 15-20%(−)-(E,R) whereas for roasted hazelnuts the ratio is about 71.5-72.5% (+)-(E,S) and 27.5-28.5% (−)-(E,R). [See Guntert, M.; Emberger, R.; Hopp, R.; Kopsel, M.; Silberzahn, W.; Werkhoff, P., Chiral analysis in flavor and essential oil chemistry. Part A, Filbertone, the character impact compound of hazelnuts, Flavor Science and Technology, Y. Bessiere & A. F. Thomas, Editors, John Wiley & Sons, Pub., Chichester, UK, pp. 29-32 (1990); Guntert, M.; Emberger, R.; Hopp, R.; Kopsel, M.; Silberzahn, W.; Werkhoff, P. Chirospecific analysis in flavor and essential oil chemistry. Part A, Filbertones, the character impact compound of hazelnuts. Z. Lebensm. Unters. Forsch., 192, 108-110 (1991); Schurig, V., Jauch, J., Schmalzing, D., Jung, M., Bretschneider, W., Hopp, R., Werkhoff, P., “Analysis of the Chiral Aroma Compound Filbertone by Inclusion Gas Chromatography”, Z. Lebensm. Unters. Forsch., 191, 28 (1990), Jauch, J., Schmalzing, D., Schurig, V., Emberger, R., Hopp, R., Köpsel, M., Silberzahn, W., Werkhoff, P., “Isolierung, Synthese und absolute Konfiguration von Filberton, dem aktiven Prinzip des Haselnuβaromas (Isolation, Synthesis, and Absolute Configuration of Filbertone, the Principal Flavor Component of the Hazelnut), Angew. Chem., 101 (8), 1039-1041 (1989); Angew. Chem. Int. Ed. Engl., 28, 1022-1023 (1989); Emberger; Roland; Kopsel, Manfred; Bruning, Jurgen; Hopp, Rudolf; Sand, Theodor; Flavoring with 5-methyl-hept-2-en-4-one, U.S. Pat. No. 4,563,365 and U.S. Pat. No. 4,654,168.]

The flavor perception threshold (in 3% sucrose solution)=0.000005 ppm for the racemic mixture and the recognition threshold is about 0.00003 ppm. At the perception threshold, (racemic) 5-methyl-hept-2-en-4-one produces a taste which can be described as “soft, buttery, full sensation in the mouth” while at and above the recognition threshold the description of the taste is “nutty, hazelnut, soft, buttery, full sensation in the mouth”. In addition to its specific characterization in the hazelnut direction in appropriate flavoring compositions, it has, in all non-nut types of compositions, a particularly rounding effect, and provides more naturalness through its buttery soft basic fullness.

The aroma and flavor characteristics of filbertone are believed to be shared by other aromachemical/flavorants wherein a double bond in the molecule is 1,4-conjugated with a keto group.

Filbertone and many of its alpha, beta-unsaturated ketone congeners suffer from being extremely volatile and water-soluble rendering them disadvantageous for use in laundry applications, such as imparting fresh, pleasant odors to clothes either through incorporation in laundry detergents, fabric softeners and the like. Immediately upon being exposed to the environment, they immediately vaporize or solubilize in the laundry water, thereby shortening their residence time on fabrics. The present inventor has found that these disadvantages can be overcome by using the compounds of the invention.

The compounds of the invention of formula (II) may be produced by the reaction of an aromachemical having at least one aldehyde or ketone group with a primary amine to form an imine of formula (II).

The aldehydes and ketones that may be used in this reaction have the formula (V):
R⁸R⁹C=O
wherein R⁸R⁹ are as defined above.

A preferred compound of formula (V) that may be used in this reaction is:

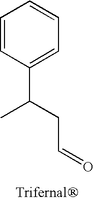

The amines that may be used in this reaction have the formula NH₂R¹⁰, wherein R¹⁰ is as defined above.

The compounds of the invention of formula (III) may be produced by a Michael addition reaction of a primary or secondary amine and an α, β-unsaturated ketone having aromachemical/flavorant characteristics of the formula (VI): 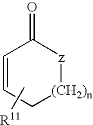
wherein Z is CH₂ or O, n is 0 or 1, such that the ring is 5- or 6-membered, respectively, and R¹¹ is as defined above.

The amines used in this reaction have the formula NHR¹R², wherein R¹ and R are as defined above.

Aromachemical/flavorants of formula (VI), which may be used in this reaction include but are not limited to:

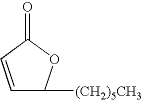

2-decen-1,4-lactone 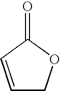
but-2-en-1,4-lactone 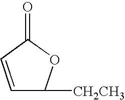
2-hexen-1,4-lactone (2(5H)-furanone)

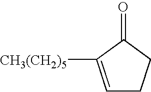

2-hexyl-2-cyclopenten-1-one (Jasmin, fruity odor, used in floral and fruity perfumes)

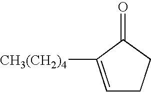

2-pentyl-2-cyclopenten-1-one (jasmine) 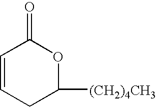
delta-2-decenolactone—(fruity, intensive peach-like, coconut, fatty, creamy)

By means of these simple, well known and conventional methods, compounds and compositions containing the compounds of the invention can be made. The reaction conditions and parameters for carrying out these procedures are well known in the prior art, although the requirements in a particular reaction for the production of optimum results may depend in each case on the reactants employed.

The perfume ingredient (ie the compound of formula (IV), (V) or (VI)) is typically used in equimolar amount to the amine compound so as to enable the reaction to take place and provide the resulting amine reaction product. Of course, higher amounts are not excluded and may even be preferred when the amine compound comprises more than one amine function. When the amine compound has more than one free primary and/or secondary amine function, several different perfume raw materials can be linked to the amine compound.

The amine compounds used in the present invention typically have an Odor Intensity Index of less than that of a 1% solution of methylanthranilate in dipropylene glycol, and a Dry Surface Odor Index of more than 5.

To measure the Odor Intensity Index, it is meant that the pure chemical is diluted at 1% in dipropylene glycol, an odor-free solvent used in perfumery. This percentage is more representative of usage levels. Smelling strips or so called “blotters”, are dipped and presented to the expert panelist for evaluation. Expert panelists are assessors trained for at least six months in odor grading and whose gradings are checked for accuracy and reproducibility versus a reference on an on-going basis. For each amine compound, the panelist is presented with two blotters: one reference methylanthranilate) and the sample. The panelist is asked to rank both smelling strips on the 0 to 5 odor intensity scale, 0 being no odor detected and 5 being very strong odor present.

Amines suitable for use in the reactions to produce the compounds of the invention are preferably non-fragrant, odorless, non-volatile amines having a relatively low vapor pressure and a relatively high molecular weight, i.e., aromatic or aliphatic amines containing more than about 10 carbon atoms containing at least one free, unmodified primary and/or secondary amino group such as, e.g., oleyl amine and the like. Preferably the amines have a molecular weight of at least 150 daltons.

Suitable alkyl or alkenyl, branched or straight chain amines that may be employed in the practice of the invention have at least about 10 carbon atoms, for example 12 to 25, or 15 to 20, for example 18 carbon atoms, are relatively odorless and form an odorless, relatively insoluble derivative with the aromachemical.

By “primary and/or secondary amine”, it is meant a component that carries at least one primary and/or secondary amine and/or amide function.

The amines and aminofunctional polymers that may be reacted with aldehydes and ketones to produce the compounds of the invention include any of those set forth in U.S. Pat. Nos. 6,699,823; 6,511,948; 6,413,920 and 6,566,312 that satisfy the criteria described herein. Moreover, the procedures described therein may be employed to prepare the compounds of the invention.

In addition, amino functional polymers containing at least one amine group may be used in the practice of the invention. The general structure for amino functional polymers containing at least one primary amine group of the present invention, is as follows:
(NH₂)ₙ-[B]
wherein n is an index of at least 1 and B is the polymer backbone. B can optionally comprise a branching group to which the NH₂ group is attached. Amino functional polymers usable in the practice of the invention include those containing a secondary amine group having a structure similar to the above excepted that the polymer comprises one or more —NH— groups instead of —NH₂ groups. Further, the polymer structure may also have one or more of both —NH₂ and —NH— groups.

The amino functional polymers that may be used in the present invention contains at least one free, unmodified primary and/or secondary amino group attached to the main chain by hydrogen substitution, or by other suitable insertion. Also suitable is the amino functional polymer comprising an unmodified primary and/or secondary amino group present on side chain(s).

Preferably, if an amino functional polymer is used it will comprise more than one amino group, more preferably more than 10 amino groups. The amino functional polymers used in the present invention will preferably have a molecular weight (MW) ranging from 400 to 100,000; more preferably up to 50,000 and still more preferably from 600 to 40,000.

The amino functional polymer can be a linear homo-, co-polymer and optionally branched, grafted and/or cross-linked.

Preferred examples of suitable amino-functional polymers for use in the present invention are selected from the polyvinylamines, derivatives thereof, copolymer thereof, alkylene polyamines, polyaminoacids and copolymers thereof, cross-linked polyaminoacids, amino substituted polyvinylalcohols, polyoxyethylene bis amines or bis aminoalkyls, aminoalkyl piperazines and derivatives thereof, linear or branched N,N′-bis-(3-aminopropyl)-1,3-propanediamine (TPTA), and mixtures thereof.

Polyamino acids are one suitable and preferred class of amino-functional polymer. Polyamino acids are compounds that are made up of amino acids or chemically modified amino acids. They can contain alanine, serine, aspartic acid, arginine, valine, threonine, glutamic acid, leucine, cysteine, histidine, lysine, isoleucine, tyrosine, asparagine, methionine, proline, tryptophan, phenylalanine, glutamine, glycine or mixtures thereof. In chemically modified amino acids, the amine or acidic function of the amino acid has been reacted with a chemical reagent. This is often done to protect these chemical amine and acid functions of the amino acid in a subsequent reaction or to give special properties to the amino acids, like improved solubility. Examples of such chemical modifications are benzyloxycarbonyl, aminobutyric acid, butyl ester, and pyroglutamic acid. More examples of common modifications of amino acids and small amino acid fragments can be found in the Bachem, 1996, Peptides and Biochemicals Catalog. Preferred polyamino acids are polylysines, polyarginine, polyglutamine, polyasparagine, polyhistidine, polytryptophane or mixtures thereof. Most preferred are polylysines or polyamino acids where more than 50% of the amino acids are lysine, since the primary amine function in the side chain of the lysine is the most reactive amine of all amino acids. The preferred polyamino acid has a molecular weight of 500 to 10,000,000, more preferably between 5,000 and 750,000.

The polyamino acid can be cross linked. The cross linking can be obtained for example by condensation of the amine group in the side chain of the amino acid like lysine with the carboxyl function on the amino acid or with protein cross linkers like PEG derivatives. The cross linked polyamino acids still need to have free primary and/or secondary amino groups left for reaction with the aromachemical ingredient. The preferred cross linked polyamino acid has a molecular weight of 20,000 to 10,000,000, more preferably between 200,000 and 2,000,000.

The polyamino acid or the amino acid can be co-polymerized with other reagents like for instance with acids, amides or acyl chlorides, for example with aminocaproic acid, adipic acid, ethylhexanoic acid, caprolactam or mixture thereof The molar ratio used in these copolymers ranges from 1:1 (reagent/amino acid (lysine)) to 1:20, more preferably from 1:1 to 1:10. The polyamino acid like polylysine can be partially ethoxylated.

Examples and supply of polyaminoacids containing lysine, arginine, glutamime, asparagine are given in the Bachem 1996, Peptides and Biochemicals catalog.

The polyaminoacid can be obtained before reaction with the aromachemical in salt form. For example polylysine can be supplied as polylysine hydrobromide. Polylysine hydrobromide is commercially available from Sigma, Applichem, Bachem and Fluka.

Examples of suitable amino functional polymers containing at least one primary and/or secondary amine group for the purpose of the present invention are:
Polyvinylamine with a MW of about from 300 to 2×10⁶;
Polyvinylamine alkoxylated with a MW of about 600, 1200 or 3000 and an ethoxylation degree of 0.5;
Polyvinylamine vinylalcohol (molar ratio 2:1), polyvinylaminevinylformamide (molar ratio 1:2) and polyvinylamine vinylformamide (molar ratio 2:1);
Triethylenetetramine, diethylenetriamine, tetraethylenepentamine;
Bis-aminopropylpiperazine;
Polyamino acid (L-lysine/lauric acid in a molar ratio of 10/1), Polyamino acid (L-lysine/aminocaproic acid/adipic acid in a molar ratio of 5/5/1), Polyamino acid (L-lysine/aminocaproic acid /ethylhexanoic acid in a molar ratio of 5/3/1) Polyamino acid (polylysine-cocaprolactam); Polylysine hydrobromide; cross-linked polylysine, amino substituted polyvinylalcohol with a MW ranging from 400 to 300,000;
polyoxyethylene bis [amine] available from e.g. Sigma;
polyoxyethylene bis [6-aminohexyl] available from e.g. Sigma;
N,N′-bis-(3-aminopropyl)-1,3-propanediamine linear or branched (TPTA); and 1,4-bis-(3-aminopropyl) piperazine (BNPP).

Preferred amino functional polymers containing at least one primary and/or secondary amine group are:
polyvinylamines with a MW ranging from 600, 1200, 3K, 20K, 25K or 50K;
amino substituted polyvinylalcohol with a MW ranging from 400 to 300,000; polyoxyethylene bis [amine] available from e.g. Sigma;
polyoxyethylene bis [6-aminohexyl] available from e.g. Sigma;
N,N′-bis-(3-aminopropyl)-1,3-propanediamine linear or branched (TPTA);
1,4-bis-(3-aminopropyl) piperazine (BNPP);
cross-linked polylysine,
Polylysine hydrobromide.

One particular object of the invention is to provide novel derivatives of aromachemicals such as alpha, beta-unsaturated aromachemical/flavorant ketones that are far less volatile and soluble than the aromachemicals themselves and that are substantive to fabrics and other substrates (ie they bind to the fabric or substrate).

The present invention provides for the use of the compounds of the invention and mixtures thereof as a flavour and/or a fragrance.

The compositions of the invention may be utilized in and for applications where there is a need for a delayed release of the odorant or flavourant. This includes compositions for use in rinses such as softening compositions, personal cleansing compositions such as shower gels, deodorants, bars, shampoos; stand alone compositions such deodorizing compositions, insecticides, and the like.

One preferred application concerns those involving contacting the compound of the invention with fabric. The compositions of the invention are suitable for use in any step, for example, of a domestic laundering operation, such as pre- and/or post-treatment compositions, as wash additives, as a composition suitable for use in the rinse process. Obviously, multiple applications can be made such as treating the fabric with a pre-treatment composition of the invention and thereafter with the composition suitable for use in the rinse process and/or drying process. By compositions suitable for use in the rinse process, these are to be understood to include compositions such as rinse added fabric softener compositions and dryer added compositions (e.g., sheets) that provide softening and/or antistatic benefits, as well as rinse additives.

The compounds of the invention typically degrade over time to release the aromachemical/flavorant slowly so that they can be enjoyed by the consumer for much longer periods of time and/or have a longer shelf-life that the parent compounds from which they are derived.

The present applicants have found that substrates may be treated with the compounds of the invention such that exposing the treated substrate to certain conditions or materials releases the flavorant/fragrance over time.

It has been found that derivatizing aromachemicals of the formula (IV) or (VI) by the 1,4- addition of a saturated or unsaturated amine to the conjugated unsaturated aldehyde or ketone leads to the production of a compound of formula (I) or (II) that has no odor or flavourant profile of its own, but which is labile to degradation to produce the original aldehyde or ketone. The same comment applies to the Schiff's bases of formula (II). Thus, the compounds of the invention have essentially the same odor characteristics as the parent compounds from which they are derived.

An example of a reaction scheme to produce a compound of formula (I), employing oleyl amine as the derivatizing agent is set forth below:

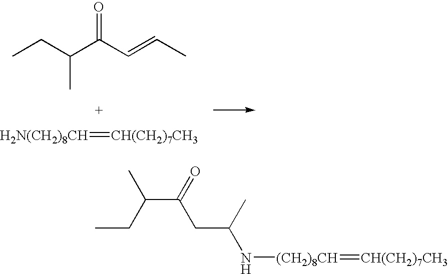

It will further be understood by those skilled in the art that the invention is also applicable to the Z isomers of filbertone and its congeners.

An example of a reaction scheme to produce a compound of formula (II), employing oleyl amine as the derivatizing agent is set forth below:

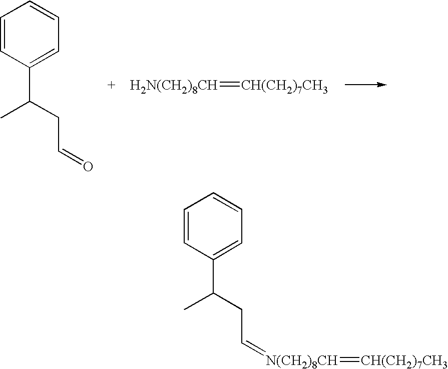

An example of a reaction scheme to produce a compound of formula (III), employing oleyl amine as the derivatizing agent is set forth below:

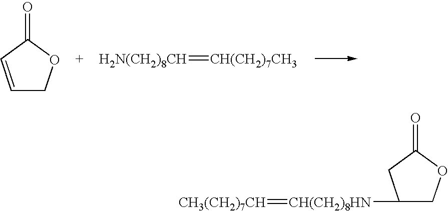

The compounds of the invention can provide fabric appearance benefits, in particular color care and protection against fabric wear. Indeed, the appearance of fabrics (e.g., clothing, bedding, household fabrics like table linens) is one of the area of concern to consumers. Indeed, upon typical consumer's uses of the fabrics such as wearing, washing, rinsing and/or tumble-drying of fabrics, a loss in the fabric appearance; which can be at least partly due to loss of color fidelity and color definition, is observed. Such a problem of color loss is even more acute after multiwash cycles. It has been found that the compositions of the present invention provide improved fabric appearance and protection against fabric wear and improved color care to laundered fabrics, especially after multiwash cycles.

Therefore, the compositions of the present invention can provide simultaneously fabric care and long lasting perfume benefits.

The compositions, products, preparations and articles in which the compounds of the invention may be incorporated include candles, air fresheners, perfumes, fragrances, colognes, soaps, bath or shower gels, shampoos or other hair care products, cosmetic preparations, body odorants, deodorants or antiperspirants, liquid or solid fabric detergents or softeners, bleach products (hypochlorites), disinfectants, all-purpose household or industrial cleaners, foods, flavorings, beverages such as beer and soda, denture cleansers (tablets), flavored orally-delivered products such as lozenges, candies, chewing gums, matrices, pharmaceuticals and the like.

The compounds can be used as perfuming ingredients, as single compounds or as mixture thereof, preferably at a range of at least about 30% by weight of the perfume composition, more preferably at a range of at least about 60% by weight of the composition. The compounds can be used in their pure state or as mixtures, without added components. The olfactive characteristics of the individual compounds are also present in mixtures thereof, and mixtures of these compounds can be used as perfuming ingredients. This may be particularly advantageous where separation and/or purification steps can be avoided by using compound mixtures.

The compounds of the invention can be included in virtually any article of manufacture that can include conventional aromachemicals, or for that matter, other fragrances, whether natural or artificial. Examples include bleach, detergents, flavorings and fragrances, beverages, including alcoholic beverages, and the like. The compounds of the invention can be used in applications like soaps, shampoos, body deodorants and antiperspirants, solid or liquid detergents for treating textiles, fabric softeners, detergent compositions and/or all-purpose cleaners for cleaning dishes or various surfaces, for both household and industrial use. Of course, the use of the compounds is not limited to the above-mentioned products, as they be used in other current uses in perfumery, namely the perfuming of soaps and shower gels, hygiene or hair-care products, as well as of body deodorants, air fresheners and cosmetic preparations, and even in fine perfumery, namely in perfumes and colognes. The compounds of the invention also find utility in foods, flavorings, beverages such as beer and soda, denture cleansers (tablets), flavored orally-delivered products such as lozenges, candies, chewing gums, matrices, pharmaceuticals and the like. These uses are described in more detail below.

In all of the applications described, the compounds of the invention can be used alone, in admixture with each other, or in admixture with other perfuming ingredients, solvents or adjuvants of current use in the art. The nature and the variety of these co-ingredients do not require a more detailed description here, which, moreover, would not be exhaustive, and the person skilled in the art will be able to choose the latter through their general knowledge and as a function of the nature of the product to be perfumed and of the desired olfactive effect.

These perfuming ingredients typically belong to chemical classes as varied as alcohols, aldehydes, ketones, esters, ethers, acetates, nitrites, terpene hydrocarbons, sulfur- and nitrogen containing heterocyclic compounds, as well as essential oils of natural or synthetic origin. A large number of these ingredients described in reference textbooks such as the book of S. Arctander, Perfume and Flavor Chemicals, 1969, Montclair, N.J., USA, the contents of which are hereby incorporated by reference in its entirety, or its more recent versions, or in other works of similar nature.

The proportions in which the conipounds of the invention can be incorporated in the various products vary within a large range of values. These values depend on the nature of the article or product that one desires to perfume and the odor effect searched for, as well as on the nature of the co-ingredients in a given composition when the compounds are used in admixture with perfuming co-ingredients, solvents or adjuvants of current use in the art.

As an example, the compounds of the invention are typically present at concentrations between about 0.1 and about 10%, or even more, by weight of these compounds relative to the weight of the perfuming composition in which they are incorporated. Far lower concentrations than those mentioned above can be used when the compounds are directly applied for perfuming the various consumer products cited beforehand.

The compounds may be used in detergents containing bleaching agents and activators such as, for example, tetraacetylethylenediamine (TAED), hypohalites, in particular hypochlorite, peroxygenated bleaching agents such as, for example, perborates, etc. The compounds can also be used in body deodorants and antiperspirants, for example, those containing aluminum salts. These embodiments are described in more detail below.

In addition to the compounds described herein, the compositions herein include a detersive surfactant and optionally, one or more additional detergent ingredients, including materials for assisting or enhancing cleaning performance, treatment of the substrate to be cleaned, or to modify the aesthetics of the detergent composition (e.g., perfumes, colorants, dyes, etc.). Non-limiting examples of synthetic detersive surfactants useful herein typically at levels from about 0.5% to about 90%, by weight, include the conventional C₁₋₁₈ alkyl benzene sulfonates (“LAS”) and primary, branch-chain and random C₁₀₋₂₀ alkyl sulfates (“AS”), and the like.

Preferred compositions incorporating only synthetic detergents have a detergent level of from about 0.5% to 50%. Compositions containing soap preferably comprise from about 10% to about 90% soap.

The compositions herein can contain other ingredients such as enzymes, bleaches, fabric softening agents, dye transfer inhibitors, suds suppressors, and chelating agents, all well known within the art.

The compounds of the invention can be incorporated into beverages and impart various flavorings to the beverages. The beverage composition can be a cola beverage composition, and can also be coffee, tea, dairy beverage, fruit juice drink, orange drink, lemon-lime drink, beer, malt beverages, or other flavored beverage. The beverages can be in liquid or powdered form. The beverage compositions can also include one or more flavoring agents; artificial colorants; vitamin additives; preservatives; caffeine additives; water; acidulants; thickeners; buffering agents; emulsifiers; and or fruit juice concentrates. Artificial colorants that may be used include caramel color, yellow 6 and yellow 5. Useful vitamin additives include vitamin B2, vitamin B6, vitamin B12, vitamin C (ascorbic acid), niacin, pantothenic acid, biotin and folic acid. Suitable preservatives include sodium or potassium benzoate. Salts that may be used include sodium, potassium and magnesium chloride. Exemplary emulsifiers are gum arabic and purity gum, and a useful thickener is pectin. Suitable acidulants include citric, phosphoric and malic acid, and potential buffering agents include sodium and potassium citrate.

The beverage may, for example, be a carbonated cola beverage. The pH is generally about 2.8 and the following ingredients can be used to make the syrup for these compositions: Flavor Concentrate, including one or more of the derivatives described herein (22.22 ml), 80% Phosphoric Acid (5.55 g), Citric Acid (0.267 g), Caffeine (1.24 g), artificial sweetener, sugar or corn syrup (to taste, depending on the actual sweetener) and Potassium Citrate (4.07 g). The beverage composition can be prepared, for example, by mixing the foregoing syrup with carbonated water in a proportion of 50 ml syrup to 250 ml of carbonated water. Flavored food and pharmaceutical compositions including one or more of the derivatives described herein can also be prepared.

The compounds of the invention can be incorporated into conventional foodstuffs using techniques well known to those of skill in the art. Alternatively, the compounds of the invention can be incorporated within polymeric particles, which can, in turn, be dispersed within and/or over a surface of an orally-deliverable matrix material, which is usually a solid or semi-solid substrate. When used in chewable compositions, the compounds of the invention can be released into the orally-deliverable polymeric matrix material as the composition is chewed and held in the mouth, thus prolonging the flavor of the composition. In the case of dried powders and mixes, the flavor can be made available as the product is consumed or be released into the matrix material as the composition is further processed. When two flavors are combined with the polymeric particles, the relative amounts of the additives can be selected to provide simultaneous release and exhaustion of the compounds.

The flavored composition can bean orally-deliverable matrix material; a plurality of water insoluble polymeric particles dispersed in the orally-deliverable matrix material, where the polymeric particles individually define networks of internal pores and are non-degradable in the digestive tract; and one or more compounds of the invention entrapped within the internal pore networks. The compounds of the invention are released as the matrix is chewed, dissolved in the mouth, or undergoes further processing selected from the group consisting of liquid addition, dry blending, stirring, mixing, heating, baking, and cooking. The orally-deliverable matrix material can be selected from the group consisting of gums, latex materials, crystallized sugars, amorphous sugars, fondants, nougats, jams, jellies, pastes, powders, dry blends, dehydrated food mixes, baked goods, batters, doughs, tablets, and lozenges.

A flavorless gum base can be combined with a compound of the invention or other suitable compound as described herein to a desired flavor concentration. Typically, a blade mixer is heated to about 110 OF, the gum base is preheated so that it is softened, and the gum base is then added to the mixer and allowed to mix for approximately 30 seconds. The flavored compound or compounds is then added to the mixer and mixed for a suitable amount of time. The gum can be then removed from the mixer and rolled to stick thickness on waxed paper while warm.

In one embodiment, the compounds of the invention are incorporated into a system that can release a fragrance in a controlled manner. These include substrates such as air fresheners, laundry detergents, fabric softeners, deodorants, lotions, and other household items. The fragrances are generally one or more derivatives of essential oils as described herein, each present in different quantities. U.S. Pat. No. 4,587,129, the contents of which are hereby incorporated by reference in their entirety, describes a method for preparing gel articles that contain up to 90% by weight of fragrance or perfume oils. The gels are prepared from a polymer having a hydroxy (lower alkoxy) 2-alkeneoate, a hydroxy (lower alkoxy) lower alkyl 2-alkeneoate, or a hydroxy poly (lower alkoxy)lower alkyl 2-alkeneoate and a polyethylenically unsaturated crosslinking agent. These materials have continuous slow release properties, i.e., they release the fragrance component continuously over a long period of time.

Advantageously, all or a portion of those compounds that include an aldehyde group can be modified to include an acetal group, which can cause the formulations to release fragrance over a period of time as the acetal hydrolyzes to form the aldehyde compound.

The present invention is illustrated by the following non-limiting examples.

### EXAMPLE 1

Equimolar amounts of amine, (267 grams of oleyl amine) and odorant (152 grams of 2-pentyl-2-cyclopenten-1 -one) were mixed by stirring thoroughly and warmed to 50° C. while stirring for 3 to 5 hours, at which time the reaction had gone substantially to completion.

The process of the reaction can be monitored by any suitable means know in the art such as NMR or chromatography eg gas chromatography or thin layer chromatography.

### EXAMPLE 2

Stoichiometric amounts of amine (35 g oleyl amine) and to filbertone (10 grams) were mixed together by stirring at about 50° C. for 3 to 5 hours, until the reaction had gone substantially to completion. When the reaction mixture had cooled (30 minutes), the product was ready for separation.

### EXAMPLE 3

The product of Example 2 was dissolved in a fabric conditioner at a concentration at which fragrances are typically found in fabric conditioners. A control fabric conditioner containing filbertone at the same concentration was also produced.

Garments were treated with the two fabric conditioners in a rinse cycle of a domestic washing machine and then dried.

It was found that garments treated with the fabric conditioner containing the product of Example 2 exhibited an odor attributable to filbertone for significantly longer than garments treated with a fabric conditioner containing filbertone itself at the same concentration. Garments treated with the filbertone containing fabric conditioner exhibited an odor attributable to filbertone for up to about one hour while garments treated with a fabric conditioner containing the product of Example 2 exhibited an odor attributable to filbertone for up to about two weeks.

Having hereby disclosed the subject matter of the present invention, it should be apparent that many modifications, substitutions, and variations of the present invention are possible in light thereof. It is to be understood that the present invention can be practiced other than as specifically described. Such modifications, substitutions and variations are intended to be within the scope of the present application.

1. A pro-odorant or pro-flavorant having the formula (I): 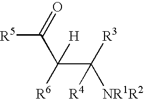
wherein R¹ is H, an aliphatic group or an aromatic group, R² is an aliphatic group or an aromatic group, provided that the total number of carbon atoms in the groups R¹ and R² is 10 or more; R³, R⁴ and R⁶ are each independently hydrogen or organic moieties which together with C(O)R⁵ render a compound of formula R⁵C(O)R⁶C=CR³R⁴ a material having odorant or flavourant characteristics and R⁵ is hydrogen or an organic moiety; or having the formula (II):
R⁸R⁹C=NR¹⁰
wherein R¹⁰ is an aliphatic group or an aromatic group having at least 10 carbon atoms; R⁸ and R⁹ are each independently hydrogen or organic moieties which together with C=O render a compound of formula R⁸R⁹C=O a material having odorant or flavourant characteristics, provided that only one of R⁸ and R⁹ is hydrogen; or having the formula (III): 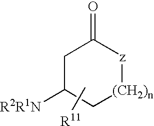
wherein R¹ is H, an aliphatic group or an aromatic group, R² is an aliphatic group or an aromatic group, provided that the total number of carbon atoms in the groups R¹ and R² is 10 or more; Z is CH₂ or O, n is 0 or 1, such that the ring is a 5 or 6 membered ring, R¹¹ is alkyl or alkenyl having up to 10 carbon atoms so as to render a compound of formula 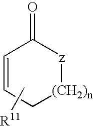
a material having odorant or flavorant characteristics.

2. A compound of formula (I) according to claim 1 having the formula:

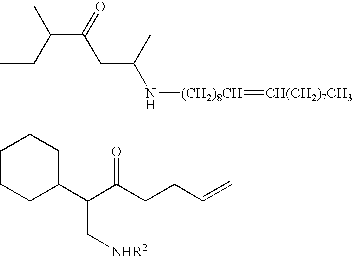

3. A compound of formula (III) according to claim 1 having the formula:

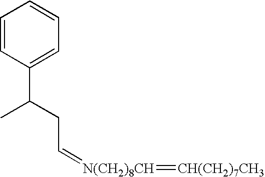

4. A compound of formula (III) according to claim 1 having the formula:

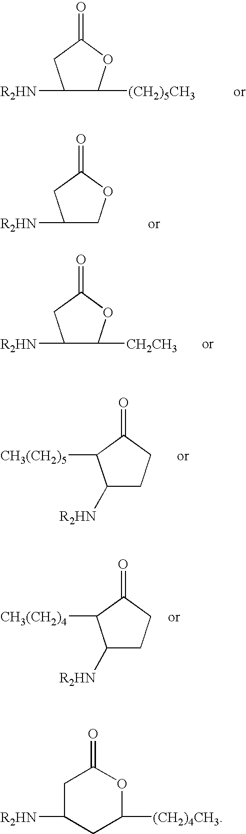

5. A compound according to claim 4, which is

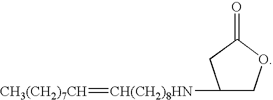

6. A process for producing a compound of formula (I) as defined in claim 1, which comprises reacting a compound of formula (IV) 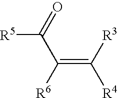
wherein R^{3,} R^{4,} R⁵ and R⁶ are each independently hydrogen or an organic moiety as defined in claim 1 with an amine of formula NHR¹R² wherein R¹ and R² are defined in claim 1; or
a process for producing a compound of formula (11) as defined in claim 1, which comprises reacting a compound of formula (V)
R⁸R⁹C=O
wherein R⁸ and R⁹ are as defined in claim 1 with an amine of formula NH₂R¹⁰,
wherein R¹ is as defined in claim 1; or
a process for producing a compound of formula (III) as defined in claim 1, which comprises reacting a compound of formula (VI) 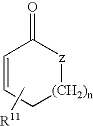
wherein Z is CH₂ or O, n is 0 or 1, such that the ring is 5- or 6-membered, respectively, and R¹¹ is as defined in claim 1 with an amine of formula NHR¹R², wherein R¹ and R² in claim 1.

7. A process according to claim 6 for producing 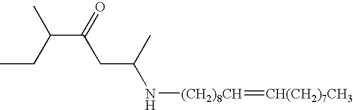
which comprises reacting

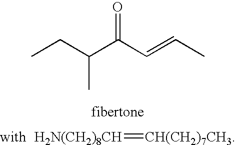

8. A process according to claim 6 for producing a compound of formula 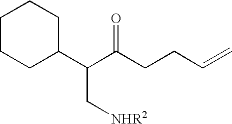
which comprises reacting a compound of formula 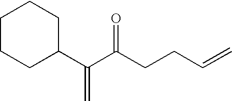
with an amine or formula NHR² wherein R² is as defined in claim 1.

9. A process according to claim 6 for producing a compound of formula 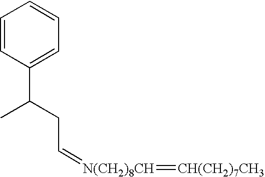
which comprises reacting a compound of formula

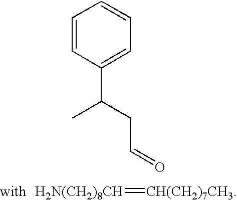

10. A process according to claim 6 for producing a compound of any one of formula 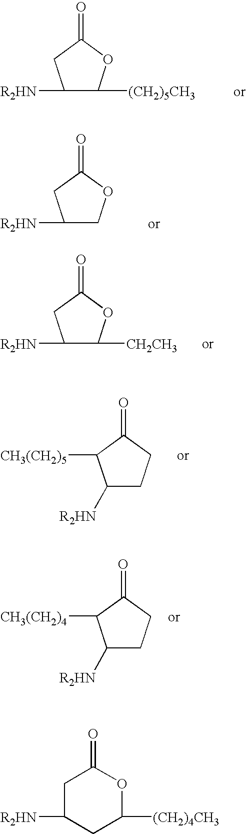
which comprises reacting the corresponding compound of formula 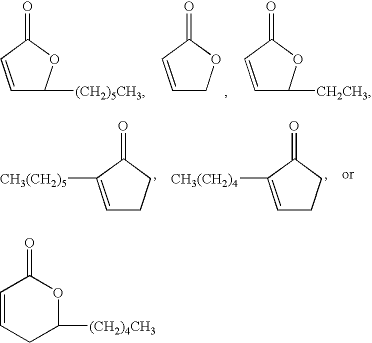
with an amine or formula NHR² wherein R² is as defined in claim 1.

11. A process according to claim 10, wherein the amine is H₂N(CH₂)₈CH=CH(CH₂)₇CH₃.

12. The use of a compound as defined in any one of claims 1 to 5 as a flavour or fragrance.

13. A substrate treated with a compound as defined in any one of claims 1 to 5.

14. A method for treating a substrate to impart flavorant/fragrance releasing characteristics thereto comprising treating the substrate with a compound as defined in any one of claims 1 to 5.

15. A composition, product, preparation or article having aroma, fragrance or odor releasing characteristics containing a compound or mixture of compounds as defined in any one of claims 1 to 5 optionally in admixture with other perfuming ingredients, solvents, or adjuvants of current use in the art.

16. A composition, product, preparation or article according to claim 15, wherein the compound or mixture of compounds as defined in any one of claims 1 to 5 is present in an amount of at least 30 percent by weight.

17. A composition, product, preparation or article according to claim 16, wherein the compound or mixture of compounds as defined in any one of claims 1 to 5 is present in an amount of at least 60 percent by weight.

18. A composition, product, preparation or article according to any one of claims 15 to 17 in the form of a perfume, fragrance or cologne, a soap, a bath or shower gel, a shampoo or other hair care product, a cosmetic preparation, a body odorant, deodorant or antiperspirant, an air freshener, a liquid or solid fabric detergent or softener, bleach product, disinfectant or an all-purpose household or industrial cleaner.

19. A detergent composition, product, preparation or article according to claim 18, wherein the compound or mixture of compounds as defined in any one of claims 1 to 5 is in admixture with other detergent ingredients, solvents or adjuvants.

20. A bleach composition, product, preparation or article according to claim 18, wherein the compound or mixture of compounds as defined in any one of claims 1 to 5 is in admixture with other bleach ingredients, solvents or adjuvants.

21. A disinfectant composition, product, preparation or article according claim 18, wherein the compound or mixture of compounds as defined in any one of claims 1 to 5 is in admixture with other disinfectant ingredients, solvents or adjuvants.

22. A composition, product, preparation or article according to claim 18 in the form of a body odorant, deodorant or antiperspirant wherein the compound or mixture of compounds is in admixture with other body odorant, deodorant or antiperspirant ingredients, solvents or adjuvants.

23. A composition, product, preparation or article having improved flavor or taste characteristics containing a compound or mixture of compounds as defined in any one of claims 1 to 5.

24. A composition, product, preparation or article according to claim 23 in the form of a beverage, which optionally contains other beverage ingredients, solvents or adjuvants.

25. A composition, product, preparation or article according to claim 23 in the form of a flavoring, which optionally contains other flavoring ingredients, solvents or adjuvants.

26. A composition, product, preparation or article according to claim 23 in the form of a food, which optionally contains other food ingredients, solvents or adjuvants.

27. A composition, product, preparation or article according to claim 23 in the form of a chewing gum, which optionally contains other chewing gum ingredients, solvents or adjuvants.

28. A composition, product, preparation or article according to claim 23 in the form of a pharmaceutical, which optionally contains other pharmaceutical ingredients, solvents or adjuvants.

29. A composition, product, preparation or article according to claim 23 in the form of an orally-deliverable matrix material which may optionally contain other matrix material ingredients, solvents or adjuvants.

30. A method to confer, improve, enhance or modify a taste or flavor property of a composition, product, preparation or article which comprises adding thereto a flavor effective amount of a compound or mixture of compounds as defined in any one of claims 1 to 5.

31. A method according to claim 30, wherein said composition, product, preparation or article is in the form of a beverage, a flavoring, a food, a chewing gum, a pharmaceutical or an orally deliverable matrix.

32. A method to confer, improve, enhance or modify an aroma, fragrance or odor characteristics of a composition, product, preparation or article which comprises adding thereto an aroma, fragrance or odor effective amount of a compound or mixture of compounds as defined in any one of claims 1 to 5.

33. A method according to claim 32 wherein said composition, product, preparation or article is in the form of a perfume, a body odorant, deodorant or antiperspirant, a detergent, a bleach product or a disinfectant.

34. An article of manufacture comprising packaging material and an aroma, odor, fragrance, taste or flavor enhancing agent contained within the packaging material, wherein the agent is effective for the enhancement of the aroma, odor, fragrance, taste or flavor of a composition, preparation, product or article to which it is added, and wherein the packaging material comprises a label which indicates that the agent can be used for enhancing aroma, odor, fragrance, taste or flavor, and wherein the agent is a compound or mixture of compounds as defined in any one of claims 1 to 5.

35. A pro-odorant or pro-flavorant as hereinbefore described.

36. A process or method as hereinbefore described.

37. A composition or article of manufacture as hereinbefore described.
